(19) United States
(12) Patent Application Publication
(10) Pub. No.: US 2021/0255157 A1
(43) Pub. Date: Aug. 19, 2021

(54) OPTIMIZED MULTI-STAGE INTERMITTENT FUGITIVE EMISSION DETECTION

(71) Applicant: SCHLUMBERGER TECHNOLOGY CORPORATION, Sugar Land, TX (US)

(72) Inventors: Kashif RASHID, Wayland, MA (US); Andrew J. SPECK, Milton, MA (US); Dominic PERRONI, Houston, TX (US); Timothy Paul OSEDACH, Medford, MA (US); Christian MEADE, Houston, TX (US); Ronald MANSON, Spring, TX (US); Andrew E. POMERANTZ, Lexington, MA (US)

(21) Appl. No.: 17/261,785

(22) PCT Filed: Jul. 19, 2019
(86) PCT No.: PCT/US2019/042522

§ 371 Date: Jan. 20, 2021

Related U.S. Application Data
(60) Provisional application No. 62/701,258, filed on Jul. 20, 2018.

(51) Int. Cl. G01N 33/00; G01N 21/39; G06Q 10/04; G01C 21/00; B64D 47/00

(52) U.S. Cl. CPC G01N 33/0047; G01N 21/39; G01N 33/0062; B64D 47/00; G06Q 10/047; G01C 21/005; G01C 21/3804; G01N 33/0075

(57) ABSTRACT

A method is provided for mitigating fugitive methane emission, which includes scanning a plurality of facilities for fugitive methane emission using an airborne sensor, and classifying the plurality of facilities based on results of the scanning. Optionally, further inspection of at least one facility of the plurality of facilities can be performed to detect and locate fugitive methane emission based on the classifying. Optionally, at least one facility can be selectively repaired based on the further inspection in order to mitigate fugitive methane emission. In another aspect, a planning workflow is provided that employs a clustering method to define cluster data representing a set of facility clusters in a geographical region that are associated with a particular base. The cluster data can be processed to determine flight path data representing flight path segments or route that form a trip, wherein the trip originates at the particular base, travels to a sequence of facility clusters and scans each facility in each facility cluster, and returns back to the particular base, wherein the sequence of facility clusters of the trip corresponds to the set of facility clusters represented by the cluster data.

### CROSS-REFERENCE TO RELATED APPLICATION(S)

The subject disclosure claims priority from U.S. Provisional Appl. No. 62/701,258, filed on Jul. 20, 2018, entitled “OPTIMIZED MULTI-STAGE INTERMITTENT FUGITIVE EMISSION DETECTION, herein incorporated by reference in its entirety.

### FIELD

The subject disclosure relates generally to detection of fugitive emissions of methane.

### BACKGROUND

Methane is the primary component of natural gas. Methane is a short-lived climate pollutant responsible for approximately twenty percent of anthropogenic greenhouse gas emissions. Fugitive methane emission can occur when methane escapes during drilling, hydrocarbon extraction, and transportation processes. Reducing fugitive methane emission in the oil and gas industry is considered among the most urgent and actionable measures to mitigate climate change, and an important complement to reducing carbon dioxide emissions.

The oil and gas industry is commonly divided into three sectors: (i) an upstream sector that finds and produces crude oil and natural gas, ii) a midstream sector that transports, stores, processes, and markets crude oil, natural gas, and natural gas liquids (such as ethane, propane and butane) as well as refined products, and iii) a downstream sector that includes oil refineries, petrochemical plants, petroleum products distributors, retail outlets and natural gas distribution companies.

Within the upstream sector of the oil and gas industry, the main technical challenge in reducing fugitive methane emission is locating methane emission sources, which typically arise from well sites or pads in remote, unmanned locations. Methane emission rates from well sites are widely distributed, with the highest-emitting 5% of sites (so called “super-emitters”) responsible for approximately 50% of fugitive methane emissions. The extent to which fugitive methane emissions can be reduced by leak detection and repair programs depends on the sensitivity of the detector used to identify methane emissions and the frequency with which inspections are performed (among other factors). Improving detector sensitivity generally results in greater methane emissions reduction because more leaks can be detected with more sensitive equipment. However, there is a threshold at which detection sensitivity is sufficient to capture all significant leaks, and further improvements in sensitivity beyond that threshold no longer result in meaningful methane emission reductions. Increasing inspection frequency generally results in greater methane emissions reduction by decreasing the duration of emission events.

Today, fugitive methane emissions in the upstream oil and gas sector are most commonly detected via optical gas imaging surveys in which a work crew drives to well sites and compressor stations and inspects for methane leaks using an infrared camera. Due to the sparse and remote locations of many sites, methane emission detection methods that involve a work crew driving to the sites are relatively inefficient.

Numerous sensors for detecting oil and gas methane emissions are being developed, including permanently installed sensors, handheld sensors, and mobile sensors mounted on trucks, drones, helicopters, airplanes, and satellites. For example, laser-based LiDAR sensors have been deployed on small aircraft. These airborne LiDAR sensors are mounted on the aircraft and employ a laser that emits a beam of electromagnetic energy that is tuned to a wavelength of strong methane absorption from the low-flying aircraft, and then detected after reflecting off the ground. This detected response can be processed to deduce the concentration of methane present in the atmosphere with a high spatial resolution. Compared to other airborne methane emissions detectors, airborne LiDAR sensors can have relatively high sensitivity, with limits of detection (determined by controlled released experiments) approaching the 1 kg methane/hour emission rate threshold under favorable conditions (i.e., wind speeds below 15 miles per hour). Airborne LiDAR technology is used today in the midstream oil and gas sector to monitor emissions from pipelines. Deploying this technology to monitor pipelines is, in one regard, relatively straightforward because the aircraft can simply fly directly along the pipeline route.

### SUMMARY

This summary is provided to introduce a selection of concepts that are further described below in the detailed description. This summary is not intended to identify key or essential features of the claimed subject matter, nor is it intended to be used as an aid in limiting the scope of the claimed subject matter.

In an embodiment, a method is provided for mitigating fugitive methane emission, which includes: scanning a plurality of facilities (e.g., well sites, compressor stations and/or other possible distributed sites of fugitive methane emission) using an airborne sensor; and classifying the plurality of facilities based on results of the scanning. Optionally, further inspection of at least one facility of the plurality of facilities can be selectively performed to detect and locate fugitive methane emission based on the classifying. Optionally, at least one facility of the plurality of facilities can be selectively repaired based on the further inspection in order to mitigate fugitive methane emission.

The method can also include building a map of the plurality of facilities.

The method can also include determining a flight path or route for the scanning. The flight path can be optimized by minimizing flight time costs for the scanning. The flight path can cover a set of facility clusters that are serviced by a respective base. The method can further include using a computer-implemented clustering method to identify the set of facility clusters that are serviced by the respective base, and using a computer-implemented vehicle routing problem (VRP) solver to determine flight path data that represents the flight path or route that covers the set of facility clusters that are serviced by the respective base as output by the clustering method. The flight path data can represent a trip that originates from the respective base and travels to a sequence of facility clusters that corresponds to the set of facility clusters and scans the facilities in each facility cluster and returns back to the respective base.

In embodiments, the airborne sensor can be a laser-based sensor, such as a LiDAR sensor. The airborne sensor can be mounted to an aircraft selected from the group consisting of a drone, a helicopter, a fixed-winged airplane, or other aircraft or flight vehicle.

In another aspect, a method is provided for planning aerial inspection of a plurality of facilities in a geographical region. The method can include storing data that represents the plurality of facilities in the geographical region and data that represents at least one base in the geographical region that supports aerial inspection of the plurality of facilities in the geographical region. A particular base in the geographical region can be selected. A clustering method can be performed on the stored data to define cluster data representing a set of facility clusters in the geographical region that are associated with the selected particular base. The cluster data output by the clustering method can be processed to determine flight path data representing flight path segments or routes that form a trip, wherein the trip originates at the particular base, travels to a sequence of facility clusters and scans each facility in each facility cluster, and returns back to the particular base, wherein the sequence of facility clusters of the trip corresponds to the set of facility clusters represented by the cluster data.

In embodiments, the data can be stored in computer memory, and the clustering method and data processing operations that determine the flight path data can be performed by at least one processor.

In embodiments, the flight path data can be determined (optimized) by minimizing flight time costs for the trip. The method can store flight vehicle data that represents operational parameters for at least one flight vehicle, and store sensor data that represents operational parameters for at least one airborne sensor. The flight time costs for the trip can be based on the flight vehicle data and the sensor data.

In embodiments, the clustering method and data processing operations that determine the flight path data can be repeated for at least one additional base in the geographic region.

In embodiments, the clustering method and data processing operations that determine the flight path data can be repeated for different combinations of flight vehicle and airborne sensor that could be used for the aerial inspection. The different combinations of flight vehicle and airborne sensor can have different flight vehicles. The different combinations of flight vehicle and airborne sensor can have different airborne sensors. The different combinations of flight vehicle and airborne sensor can also have both different flight vehicles and different airborne sensors.

In embodiments, the method can further include using the flight path data to determine overall costs for the different combinations of flight vehicle and airborne sensor and evaluating the overall costs for the different combinations of flight vehicle and airborne sensor in order to select a particular combination of flight vehicle and airborne sensor that will be used for the aerial inspection. The overall costs for the different combinations of flight vehicle and airborne sensor can be based on financial parameters for the different combinations of flight vehicle and airborne sensor.

In embodiments, the method can further include using the particular combination of flight vehicle and airborne sensor and the flight path data for the particular combination of flight vehicle and airborne sensor to perform the aerial inspection of the facilities in the geographical region.

In embodiments, the clustering method can be a hierarchical multilevel clustering method.

In embodiments, the clustering method can be applied to a filtered set of facilities that are associated with the particular base.

In embodiments, the data processing operations that determine (optimize) the flight path data can use a computer-implemented vehicle routing problem (VRP) solver to determine the flight path data. The VRP solver can employ a graph with the facility clusters defined as vertices of the graph, time to travel between clusters at flight vehicle cruising speed defined as edge costs in the graph, scan times for scanning each facility in a respective cluster embedded as vertex costs in the graph, and vehicle range limits imposed as capacity constraints. No-fly zone restrictions and possibly other limitations can be defined by a set of constraints that are added as penalties on non-compliant edges of the graph.

In embodiments, the method can further include storing data representing a template scan pattern which is intended to be used in scanning the one or more facilities in a respective cluster. The flight time costs for a trip can include scanning costs for scanning the respective cluster which is based on the data representing the template scan pattern. Such scanning costs can be further based on parameters of a bounding box that covers the one or more facilities in the respective cluster.

In other embodiments, the flight time costs for a trip can include scanning costs based on optimization of the flight pattern for the one or more facilities of the respective cluster that minimizes flight times for scanning the one or more facilities of the respective cluster.

In embodiments, the method can further include storing data representing flight vehicle scan speed which is intended to be used in carrying out scanning one or more facilities in a respective cluster. The flight time costs for a trip can include scanning costs based on flight vehicle scan speed.

In embodiments, the method can further include storing data representing flight vehicle cruise speed. The flight time costs for a trip can be based on the flight vehicle cruise speed for the flight segments or routes of the trip between the base to the sequence of facility clusters, between facility clusters, and back to the base.

In embodiments, the flight time costs for a trip can be based on at least one operational parameter of an airborne sensor. For example, the at least one operational parameter can be selected from the group consisting of scan swath, scan speed, scan radius, weight, cost, deployment restrictions, and possibly other parameters. The airborne sensor can be a laser-based sensor, such as a LiDAR sensor.

In embodiments, the flight time costs for a trip can be based on at least one operational parameter of a flight vehicle. For example, the at least one operational parameter can be selected from the group consisting of cruise speed, fuel burn rate, fuel capacity, turn rate, and possible other operating limits. The flight vehicle can be selected from the group consisting of a drone, a helicopter, a fixed-winged airplane, or other aircraft or flight vehicle.

A data processing apparatus that includes computer memory and at least one processor can be configured to carry out parts or all of the planning operations for aerial inspection of a plurality of facilities (such as well sites, compressor stations and/or other possible distributed sites of fugitive methane emission) in a geographical region to detect fugitive methane emission.

Other aspects are also described and claimed.

### BRIEF DESCRIPTION OF THE DRAWINGS

The subject disclosure is further described in the detailed description which follows, in reference to the noted plurality of drawings by way of non-limiting examples of the subject disclosure, in which like reference numerals represent similar parts throughout the several views of the drawings, and wherein:

FIGS. 1A-1C, collectively, is a flowchart that illustrates an exemplary workflow of the subject disclosure;

FIG. 2 illustrates an example flight path planning solution produced by the workflows of the subject disclosure as well as a template scan path for scanning one or more facilities of the clusters produced by the workflows; and

FIG. 3 illustrates an example computing device that can be used to embody parts of the workflow of the present disclosure.

### DETAILED DESCRIPTION

The particulars shown herein are by way of example and for purposes of illustrative discussion of the examples of the subject disclosure only and are presented in the cause of providing what is believed to be the most useful and readily understood description of the principles and conceptual aspects of the subject disclosure. In this regard, no attempt is made to show structural details in more detail than is necessary, the description taken with the drawings making apparent to those skilled in the art how the several forms of the subject disclosure may be embodied in practice. Furthermore, like reference numbers and designations in the various drawings indicate like elements.

With regard to the embodiments of the workflows described herein that deploy airborne sensors to monitor and detect fugitive methane emissions in the upstream oil and gas sector, the term “airborne sensor” or “sensor” refers to a mobile instrument or apparatus that is mounted to a flight vehicle and that can be configured to monitor and detect fugitive methane emissions originating from surface-located facilities from the air while flying the flight vehicle. In non-limiting examples, an airborne sensor can be a LiDAR instrument, a gas remote detection instrument, a differential-absorption LiDAR instrument, a gas-mapping LiDAR instrument, a laser-based detection instrument, a non-laser-based detection instrument e.g. a spectrometer, or other suitable remote methane sensor. Note that the swath, scanning speed, sensitivity and other operational parameters can vary amongst the different types of airborne sensors.

The term “flight vehicle” refers to a vehicle that is capable of travelling through the air. In non-limiting examples, a flight vehicle can be a drone, helicopter, a fixed-winged airplane, or other aircraft or flight vehicle.

The term “base” refers to a physical location from which a flight vehicle and airborne sensor combination is deployed to initiate a flight that performs airborne inspection of a sequence of one or more facilities. In non-limiting examples, a base can be an airport or landing strip or other suitable locations from which a flight vehicle with airborne sensor can be deployed.

The subject disclosure describes workflows that deploys airborne sensors to monitor and detect fugitive methane emissions in the upstream oil and gas sector. Deploying the airborne sensors in the upstream oil and gas sector is challenging because of the complex and sparse arrangement of upstream oil and gas facilities such as well sites and compressor stations. Comprehensive and cost-effective monitoring and detection of fugitive methane emissions in the upstream oil and gas sector using airborne sensors therefore requires an efficient deployment scheme. The subject disclosure provides a workflow that generates an optimized deployment scheme for the use of airborne sensor technology in monitoring and detecting fugitive methane emissions in the upstream oil and gas sector. The workflow can also be extended to estimate the environmental benefits and implementation costs associated with the optimized deployment scheme.

In embodiments, the workflow can involve a multi-stage measurement scheme. In the first stage, one or more airborne sensors are used to monitor and detect methane emissions from upstream oil and gas facilities (e.g., well sites, compressor stations and/or other possible distributed sites of fugitive methane emission). The results of such monitoring and detection operations are used to classify locations where an airborne sensor has detected methane emissions and locations where an airborne sensor has not detected methane emissions. This first stage is optimized by a procedure designed to manage facility visits in an optimal manner (for example, with respect to choice of flight vehicle, airborne sensor and base). In a second stage, locations where an airborne sensor has detected methane emissions in the first stage can be subjected to a more precise but more expensive component-level inspection and repair, if need be. The component-level inspection and repair can involve inspection and repair of valves, flanges, tanks or other equipment or other components of a facility. The addition of the optimized first stage is intended to lower the cost of the component-level inspection of the second stage relative to the current practice of inspecting all well site and compressor station locations at the component level.

Component-level facility inspections typically require a small team to spend hours inspecting a facility (and often to spend hours driving to-and-from the location). To make the inspection process more efficient and less expensive, the workflow of the subject disclosure monitors and detects methane emissions from the facilities using airborne sensor technology. The route traveled by the flight can be generated by computer-implemented optimized procedures that are configured to manage facility visits in an optimal manner (for example, with respect to choice of flight vehicle, sensor and base). Using this optimized deployment scheme, the inspection time per facility can be reduced from hours to minutes.

Note that the airborne sensor can typically determine the presence of methane emission at a facility that is sufficiently large as to require repair, but it cannot identify the location of the methane emission (or leak) with sufficient precision as required to repair the leak, while traditional manual inspection using portable detectors will provide sufficient precision. Thus, in the workflows described here, the facilities that are identified by the airborne sensor inspection to have fugitive methane emissions can be subject to a second component-level inspection and repair. The component-level inspection and repair can involve inspection and repair of valves, flanges, tanks or other equipment or other components of a facility. Such component-level inspection and repair can possibly use traditional manual inspection and repair methods. Because the workflows described herein limit the component-level inspection operations only to locations that are determined to be leaking from the inexpensive optimized airborne inspection, the total cost of inspection is lower than for the traditional procedure where the component-level is performed on all locations (or for other procedures in which the initial inspection is performed in a less efficient manner).

In embodiments, the workflow as described herein deploys airborne sensor technology to rapidly scan multiple facilities for fugitive methane emissions. The scanning takes place in multiple stages. In this first stage, one or more airborne sensors are used to rapidly scan multiple facilities for fugitive methane emissions. The results of the scanning process are used to classify locations where an airborne sensor has detected methane emissions and locations where an airborne sensor has not detected methane emissions. In a second stage, one or more facilities where an airborne sensor has detected methane emissions in the first stage are inspected for fugitive emissions with slower but more precise technology in which the presence of fugitive emissions is confirmed and the location of the fugitive emissions is identified and possibly repaired, if need be.

In one embodiment, a workflow that deploys airborne sensors to monitor and detect fugitive emissions in the upstream oil and gas sector employs the following operations:

i) Build a map of the locations of facilities (such as well sites, compressor stations, or other distributed sources of methane emission) to be scanned. This information can be obtained directly from an oil and gas company interested in having their facilities monitored for fugitive emission, from a database, or from another source.

ii) For each facility to be scanned, determine the area near each facility that requires scanning. This area may be offset from the center of the facility in the direction of prevailing winds at the intended time of the survey. This area may be larger than the area of the facility to account for atmospheric gas dispersion beyond the area of the facility.

iii) Build a map of the locations of one or more bases.

iv) Optionally, collect and collate data describing costs and specification details of available vehicles and sensors.

v) Execute a computer-implemented optimization procedure that is configured to plan and manage designated facility visits in an optimal manner with respect to choice of flight vehicle, sensor and base. The outcome of this procedure will be a collection of trips (flight path segments or routes) that serve to optimally scan the area associated with all the facilities of the data set for methane emission detection. This procedure is described in further detail below.

vi) The results from the preceding block v) are used to identify the least costly flight vehicle and sensor (or flight vehicle-sensor combination) amongst the set of available flight and vehicle-sensor combinations under consideration. Note that the optimization process can be repeated for the best combination flight vehicle and sensor using finer parameterization to furnish the best possible flight paths prior to implementation.

vii) The flight vehicle and sensor identified in block vi) can then be used to scan the designated facilities using the collection of flight paths produced in block v) or vi).

viii) Facilities where the scan results of the airborne sensor detect methane emission can be marked during, or after the scan, as requiring further inspection to validate methane emissions.

ix) For facilities where a further component-level inspection is deemed necessary by the previous block viii), optionally compare the time of airborne sensor scanning to the time of any activities that may result in temporary emissions, such as liquid unloading. The airborne sensor scanning can then be repeated at facilities where potential false positive reports may have occurred due to activities resulting in temporary methane emissions.

x) Schedule component-level inspection and repair for facilities that are deemed to require a further inspection after block ix).

xi) Perform the component-level inspection scheduled in block x) to detect and locate fugitive methane emissions at the respective facilities. The component-level inspection can involve inspection of valves, flanges, tanks or other equipment or other components of the respective facilities. In embodiments, the component-level inspection can utilize portable technology that can effectively identify emissions, such as a gas sniffer or an optical gas imager.

xii) Repair facility components and equipment that produce the methane emissions identified in block xi), for example using standard best practices.
xiii) Verify the quality of the repair of block xii) by inspecting the repaired facility components or equipment. In embodiments, portable technology as described above in block xi) can be used in block xiii) for validation of leak mitigation.

### Optimal Flight Path Planning Workflow

Block v) of the workflow outlined above is a computer-implemented optimization procedure that serves to establish flight vehicle routes necessary to carry out aerial inspection (or scanning) of a set of desired facilities (such as well sites, compressor stations, or other distributed sources of methane emission). The routes can be traveled by one or more flight vehicles in order to carry out the aerial inspection. The operations associated with this procedure are described in greater detail below:

1(a)—Define a set of facilities (such as well sites, compressor stations, or other distributed sources of methane emission) to be scanned as the data of interest.

1(b)—Define a set of flight vehicles under consideration.

1(c)—Define a set of airborne sensors under consideration.

1(d)—Define a set of bases (and corresponding base locations) from which scanning of the facilities in the data set of 1(a) can be initiated.

2(a)—Define key attributes for each well site in the data set of 1(a); for example, such key attributes can include, but are not limited to, location, scan radius, center offset, etc.

(2b)—Define key attributes for each flight vehicle in the data set of 1(b); for example, such key attributes can include, but are not limited to, cruise speed, energy consumption rate, energy capacity, operating limits, etc.; note that such key attributes can be used to establish vehicle operating range in both distance and time.

(2c)—Define key attributes for each sensor in the data set of 1(c); for example, such key attributes can include, but are not limited to, sensor swath, sensor scan speed, etc.

(2d)—Define key attributes for each base in the data set of 1(d); for example, such key attributes can include, but are not limited to, resources available, vehicle operating restrictions and facilities, etc.

(2e)—Define restrictions (constraints) for the given model data; for example, such restrictions can include, but are not limited to, no-fly zones, operating restrictions, safety measures, operator selection, etc.

(2f)—Define a set of permissible vehicle, sensor and base combinations for the given data.

(3a)—Select a vehicle, sensor and base combination from the set of combinations defined in (2f).

(3b)—Execute an optimization routine to establish a time-distance solution defining an optimal number of trips with routes for the vehicle, sensor and base combination selected in (3a).

(3c)—Apply financial parameters (stemming from operator practices or due to prevailing cost models) to the time-distance solution produced by block (3a) above. The complete solution can be stored in a table or other computer data structure and can be used later for comparative purposes.

(4) Repeat the operations of blocks (3a) and (3b) and (3c) for additional vehicle, sensor and base combinations in the set of combinations defined in (2f).

(5) When all of the vehicle, sensor and base combinations in the set of combinations defined in (2f) have been processed, continue to block vi) of the workflow described above.

### Optimizations Employing Hierarchical (Multi-Level) Clustering and Vehicle Routing Problem (VRP) Solver

In embodiments, the optimization routine of block (3b) uses a hierarchical (multi-level) clustering method to group the facilities into one or more clusters of facilities that are associated with the particular base of the vehicle-sensor-base combination under consideration. As the number of clusters cannot be known a priori, the routine can be applied by iteration.

At each iteration, any number up to the maximum designated clusters can be identified. The effective scan area of each cluster can be evaluated and any cluster that exceeds a distance limit (or time limit) of the designated vehicle-sensor combination can be flagged for subsequent sub-clustering. Subsequently, second-level clustering ensures that each identified cluster group is within operating limits of the designated vehicle-sensor combination. In other words, if a flight vehicle arrives at any target site (a cluster center), it will be able to perform the scan of the one or more facilities of the cluster within operating limits. Note that the clustering method can identify the location of the centers of the clusters. Each facility within a cluster can be assigned an error measure based on least distance to the cluster center.

The clusters generated by the second-level clustering represent groups of facilities in the absence of any designated base. Thus, in a third-level clustering, each facility within a cluster is evaluated with respect to the base location for the particular base of the vehicle-sensor-base combination under consideration and marked as either feasible or infeasible. A feasible cluster is one that can be reached from the stipulated base location, permits scanning of all the facilities of the cluster as per requirements by cluster size (given by the underlying facilities and resulting scan area), and finally ensures that the flight vehicle is able to return to the stipulated base location, all within safe operating margins. Any cluster that does not satisfy the constraints of the feasible cluster is marked as an infeasible cluster. The third-level clustering can then be reapplied to any infeasible cluster resulting in sub-cluster groups, possibly, down to an individual facility, if necessary. Those facilities that cannot be reached are discarded as ‘unattainable’ by definition for the vehicle-sensor-base combination under consideration.

Furthermore, the feasible clusters can be parsed by some user-defined measure (e.g., as a function of site scan area, well density, or some other measure) to enforce a further sub-clustering requirement. When the hierarchical clustering process completes, it will result in a set of desired and feasible facility clusters for the given vehicle-sensor-base combination under consideration, and no further clustering levels are warranted.

In embodiments, the effective cluster center for the feasible facility clusters can be calculated. For example, the effective cluster center for a given facility cluster can be derived as the center-of-mass of the facilities that belong to the given cluster. This ensures that the cluster center resides within the scan area in case of sub-optimality in the clustering procedure.

The result of the hierarchical clustering method is data that represents a set of clusters of associated facilities for the given vehicle-sensor-base combination under consideration. These results, together with the data representing flight vehicle, sensor and base combination, results in a vehicle routing problem (VRP). That is, how many trips are required from the given starting location of the base to serve each facility belonging to the set of clusters and then returning to the same base location. Note that a dedicated VRP solver can be used to address this problem with vehicle range limits imposed as capacity constraints. The anticipated costs can be embedded as costs in the VRP graph with respect to the end node in the given leg. Similarly, no-fly zone restrictions can be added directly as penalties to the non-compliant edges in the graph at the outset. The VRP solver then will yield the optimal number of trips along with their anticipated routes to minimize the overall time or distance measure (as a cost of the entire process). Note that as a flight vehicle is deemed to travel to a cluster at cruise speed but undertakes scan operations of the one or more facilities of the cluster at scan speed, cumulative time is a good measure to use that also allows ready consideration of vehicle total hire time. However, distance, or some other metric, could also be used for performance purposes.

With respect to the optimization routine of block (3a) described above, several points are worthy of elaboration.

First, a large dataset (e.g., one comprising tens of thousands of well sites) necessarily leads to a great computation cost and effort in establishing a clustering and routing solution, as per the method described above. Thus, it can be expedient to partition the facilities of the dataset by assignment to the nearest base location a priori. However, if the resulting data set is still very large, a spatial partitioning procedure can be applied within the locality of the given base. That is, the facilities can be sub-partitioned by quadrant or more generally, by some fraction of the angle between set bounds, that includes the density measure of the facilities held within each region. Each sub-problem can be solved independently, with the collective solution given by the set of all sub-solutions for that given base.

In some instances, partitioning facility data by assignment to the nearest base can be inefficient if certain bases result in the assignment of a few facilities. This means that in the operational implementation, the vehicle and crew must move to a new base (at some cost) to target the remaining facilities. However, rather than incur this cost, it may be more conducive (economic) to fly from a more heavily-used base, albeit with longer flight incursions. In that regard, an alternative procedure can be used whereby a base is selected in order of facility assignments, and all facilities that can be reached from that base are completed before moving to the next base on the list. For bases that must be used, the facilities can be re-assigned by nearest base, while those bases which had a few target facilities that were successfully fielded by a more significant base location can now be omitted from the planning process. The plans should be re-optimized for the set of selected bases with facility assignment to the nearest base location.

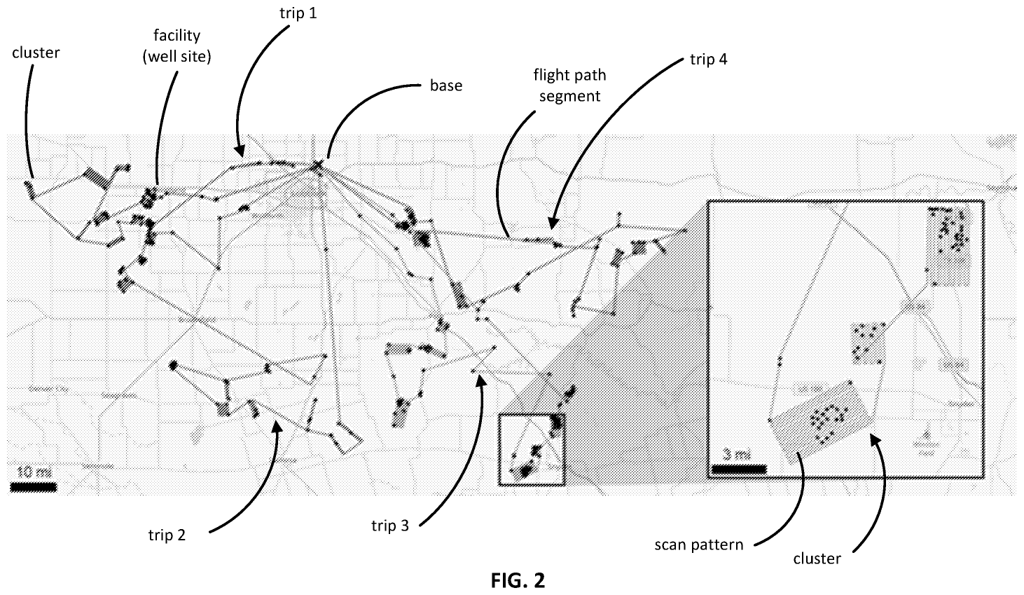

Lastly, it should be clear that the clusters can include a number of underlying facilities. The area defined by this collection dictates the scan area of the cluster. The optimization problem then involves establishing a flight pattern to cover the scan area of each cluster. This could be done directly by solving a cluster cover optimization problem at each-and-every cluster or more expediently, using a template design that provides a quick solution. The latter involves the use of a set flight pattern (or template scan pattern) around the facilities of the cluster such that the designated scan area is implicitly covered including all desired facilities of the cluster as shown in FIG. 2. The template scan pattern may not be as efficient as a rigorous site optimization scheme due to the distribution of facilities, i.e., the flight pattern may unnecessarily, and undesirably, include dead-space where no facilities are located. This issue can be mitigated by limiting the maximum scan area to some extent. Nonetheless, the advantage of using the template scan pattern is fast computation, along with the fact that the template scan pattern is more likely to be used in practice. For example, a “wing-over” template scan pattern in which the pilot flies linearly over a rectangular field but makes a fast-rising pull-out turn to the right before performing an altitude dropping 180 degree turn to get back in-line with the field on the return pass. This procedure can be repeated until the rectangular field has been fully scanned (or sprayed) over multiple passes as shown in FIG. 2. Similarly, another type of template scan pattern can use the notion of hair-pin turns at fixed altitude, but with the same intention to cover a rectangular field with the fewest number of passes. The workflows described herein may use any given template scan pattern design, or undertake a rigorous site optimization, such that the time and distance values to complete the site scan over the designated area (encompassing all underlying facilities) are provided as an outcome. These measures are anticipated by the hierarchical clustering method and consequently are used in the vehicle routing problem as described above.

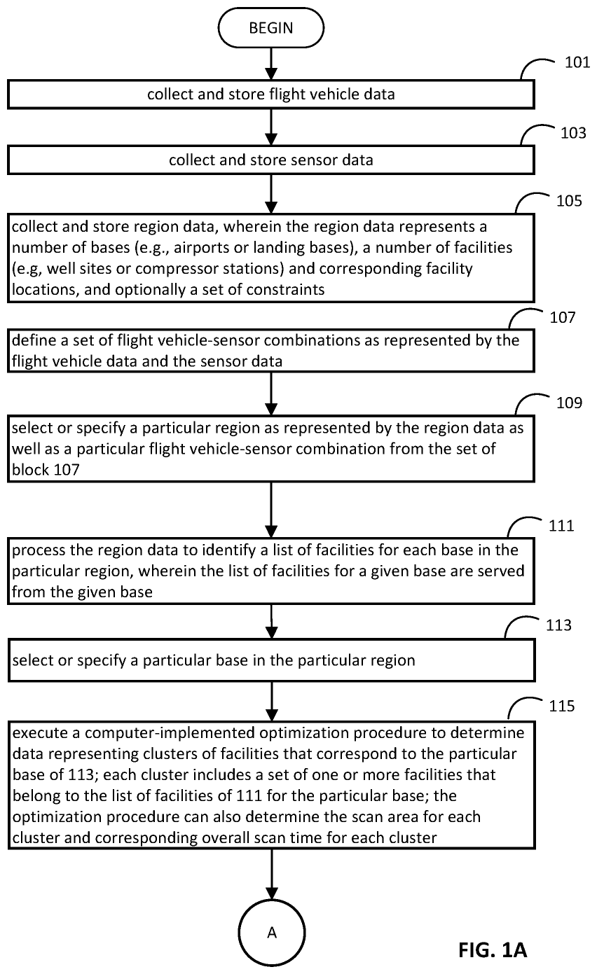

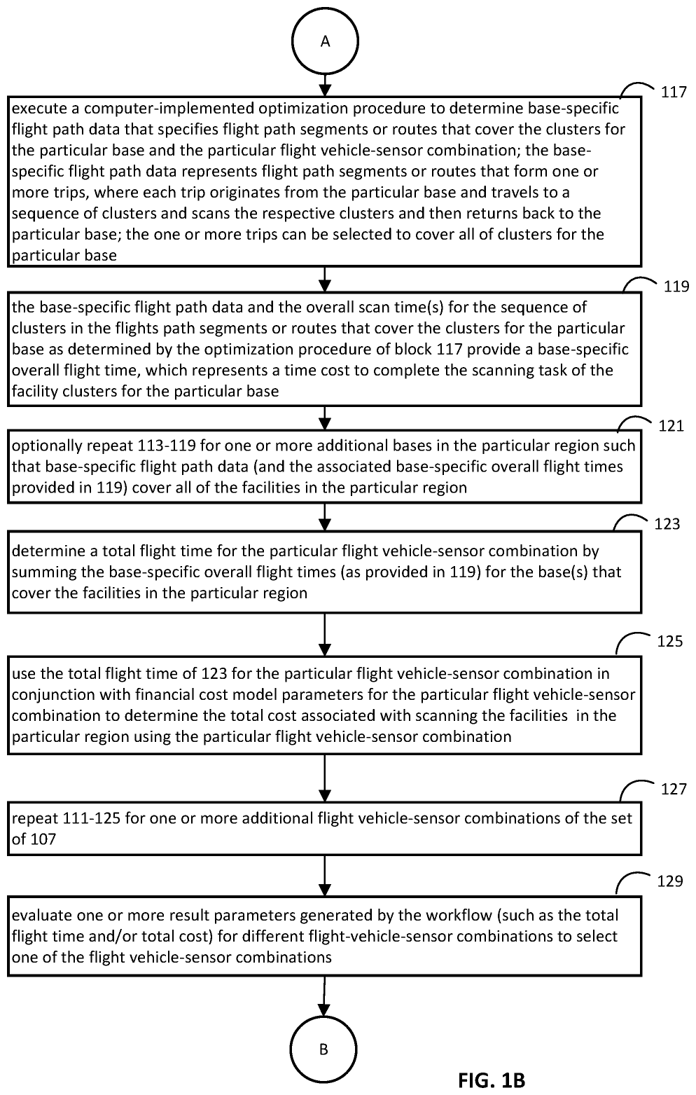

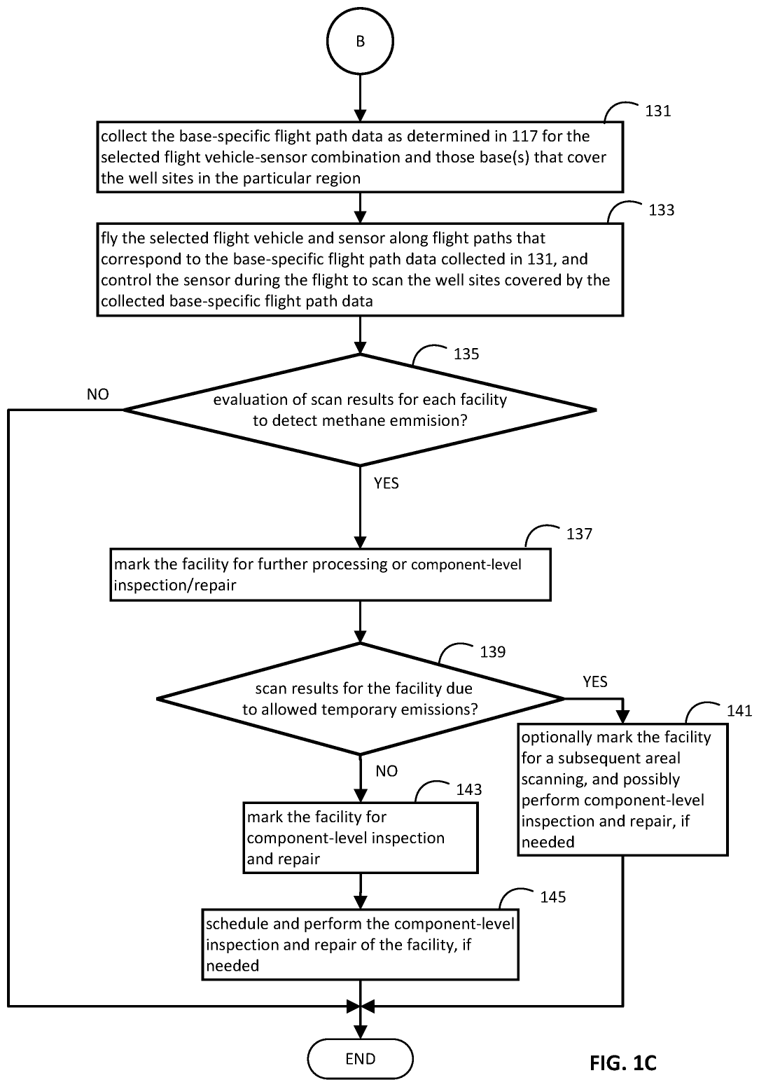

FIGS. 1A-1C is a flowchart that illustrates another exemplary workflow that deploys airborne sensors to monitor and detect fugitive emissions in the upstream oil and gas sector.

In block 101, flight vehicle data can be collected and stored. The flight vehicle data can represent operational parameters for one or more flight vehicles. For example, the flight vehicle data can define a set of vehicles V, where a particular vehicle V includes the following parameters: name, cruise speed (kmph), fuel burn rate (per hour), fuel capacity, turn rate (hours), and possible other operating limits.

In block 103, sensor data can be collected and stored. The sensor data can represent operational parameters for one or more airborne sensors. For example, the sensor data can define a set of sensors S, where a particular sensor S includes the following parameters: name, scan swath (km), scan speed (kmph), scan radius (km), weight, cost, deployment restrictions (such as wind speed), limit of detection, and possibly other parameters.

In block 105, region data can be collected and stored. The region data represents a number of bases (e.g., airports or landing bases), a number of facilities (e.g., well sites, compression stations, and/or other distributed upstream facilities that are potential sources of methane emission) and corresponding facility locations, and optionally a set of constraints. For example, the region data can define a set of regions R, where a particular region R comprises the list of all facilities F in the region, a list of available bases B in the region, and a set of constraints C for the region. Each well F in F can include a unique identification number for the facility and a location for the facility in the cartesian coordinate system of R. Similarly, each base B in B can include a name, location and possible operating limits. The set of constraints C defines no fly-zones, restrictions, or other operating limitations in R, where each constraint C in C can be expressed as an exclusion by rectangular, circular or linear defined bounds.

In block 107, a set of possible flight vehicle-sensor combinations is defined according to the flight vehicle data and the sensor data. For example, a set of possible vehicle-sensor combinations U can be defined, where a particular vehicle-sensor combination U comprises a valid vehicle V and sensor S pair.

In block 109, a particular region as represented by the region data as well a particular flight vehicle-sensor combination of the set of block 107 are selected or specified. Such selections can be based on user input or automatically by software instructions.

In block 111, the region data can be processed to identify a list of facilities for each base in the particular region of 109, wherein the facilities for a given base are served from the given base. In embodiments, the processing of block 111 can involve using the region data collected and stored in block 105 to initialize a set of facilities F, a set of bases B and a set of constraints C for a region R as selected in 109. The set of facilities F can be filtered according to an operator selection list to give a filtered set of facilities F_{f}. This set is further filtered for each base B in B, giving a set of facilities F_{B} that include those facilities that are located nearest to B and should therefore be preferentially served from that base B. For a very dense data-set, the set of facilities F_{B} can be further partitioned by quadrant (or some other means) yielding a collection of sets {F_{B1}, F_{B2}, . . . , F_{Bk}} for k E {1, . . . , K} that are managed from the base B, collectively ensuring that all (reachable) facilities in F_{B} are covered.

In block 113, a particular base that is located within the particular region of 109 is selected or specified. Such selection can be based on user input or automatically by software instructions.

In block 115, a computer-implemented optimization procedure is executed to determine data representing clusters of facilities that correspond to the particular base of 113. Each cluster includes a set of one or more facilities that belong to the filtered set of facilities of 111 for the particular base. The optimization procedure can also determine the scan area for each cluster and corresponding overall scan time for each cluster.

In embodiments, the optimization procedure of block 115 is performed for a given vehicle V, sensor S, base B, set of facilities F_{Bk} (specified generally as D) for the base B, and the set of constraints C as follows. First, a clustering procedure is applied to the set D to identify a number of facility clusters (or target-sites). As the number of anticipated clusters is not known a priori, the procedure is applied for a given number of clusters (n) as follows:

$$\begin{matrix}min & M ( X \vert D ) \\ s . t & {d}_{min} - ‖ {x}_{i} - {x}_{j} ‖ \leq 0 \\ & {x}_{L} \leq {x}_{i} , {x}_{j} \leq {x}_{U} \\ & i , j = \{ 1 , 2 , … , n \}\end{matrix} \qquad Eqn . ( 1 )$$

where, X is the set of clusters (ϵ [symbol] ²), dₘᵢₙ is the minimum permissible distance between any two cluster centers xᵢ and xⱼ (ϵ [symbol] ²) with lower and upper bounds x_{L} and x_{U} respectively, and M is the collective measure of total distance of each of the m samples dⱼ in D (with j={1, 2, . . . , m}) to its nearest cluster center, xⱼ^{min}=min{∥dⱼ−xᵢ∥} for all i={1, 2, . . . , n}, defined as:

M(X|D)=Σⱼ₌₁^{m}∥dⱼ−xⱼ^{min}∥ Eqn. (2)

The set of candidate clusters X is filtered of any clusters with zero facility assignments to give the set of target clusters C_{L} of size c.

Then for each cluster in the set C_{L}, the cluster center is determined as the center of mass of the prevailing sample set d (those wells assigned to the cluster). In addition, the lower-left and the upper-right points that define the bounding set of the facilities in the cluster (including a buffer in consideration of site scan radius) is used to estimate the scan area of each cluster in the set C_{L}. The center-of-mass of a given cluster in the set C_{L} can be determined in the cartesian XY coordinate system of the particular region in which they are located. Specifically, the X-coordinate of the cluster center of mass can be determined by dividing the sum of the X-coordinates of the facility locations of the cluster by the number of facility locations in the cluster, and the Y-coordinate of the cluster center of mass can be determined by dividing the sum of the Y-coordinates of the facility locations of the cluster by the number of facility locations in the cluster.

The width (lₓ) and height (l_{y}) of the bounded region, along with the properties of the vehicle V (Cruise Speed, Turn Time) and sensor S (Scan Swath and Scan Speed) can then be used to infer the time required to scan each cluster (in hours):

Tₛ=f(lₓ,l_{y},V,S) Eqn. (3)

Importantly, as the regular bound does not ensure least area, the bound set is optimized to give the minimum expected scan time, defined as follows:

$$\begin{matrix}min & S ( P , Q , w \vert d ) \\ s . t & point {d}_{i} is within bounds ( P , Q , w ) \\ & distance of {d}_{i} to nearest point on each bound > \\ & Site Radius \\ & i = \{ 1 , 2 , … , I \} is the index of facilities in d \\ & {x}_{L} \leq P , Q \leq {x}_{U} are points ( {ϵℝ}^{2} ) in region R \\ & {w}_{min} \leq w \leq {w}_{max} where , {w}_{min} = Site Radius ( km )\end{matrix} \qquad Eqn . ( 4 )$$

Here, the control variable set {P, Q, w} (ϵ [symbol] ⁵) defines the location of a point P that connects to a point Q with orthogonal bounds of width w. Hence, P, Q and w, define a bound set (points P, Q, R and S) around the facilities d in the given cluster C. The solution of this problem is the least scan time required to cover the bound set defined by points P, Q, R and S. This procedure is applied to each one of the c clusters in C_{L}. That is, each cluster in C_{L} has a designated site scan cost in terms of time (hours) once evaluated. This information is important, as subsequently the costs (of each cluster group) can be imposed as the target node costs in the vehicle routing problem (block 117).

Any cluster that exceeds the vehicle-sensor imposed area or time limit can be flagged for further sub-clustering. That is, a second-level of clustering can be applied to ensure that each identified cluster is within operating limits of the vehicle V-sensor S combination. That is, if the vehicle V arrives at the target site (a cluster center), it can perform the scan of the facilities of the cluster within its operating limits and return to the base, e.g., the time to travel to-and-from the base to the cluster center plus site scan cost must be less than Tₘₐₓ, the maximum vehicle flying time (fuel capacity divided by fuel burn rate). This second level of clustering can be performed on the cluster groups using the same procedure described above. The final set of target clusters C_{L} of size c is updated accordingly.

Note that zero or more facilities that cannot be reached within the operational constraints of the vehicle V-sensor S combination under consideration can be marked as ‘unattainable’ and discarded from the facilities that will be scanned. In this case, each unattainable facility can be inspected by other methods, such as by a physical inspection similar to block 145 as described below. If this inspection detects and locates fugitive methane emission, the location of the leak can be repaired as described in block 145 below.

In block 117, a computer-implemented optimization procedure is executed to determine base-specific flight path data that specifies flight path segments or routes that cover the facility clusters for the particular base of 113 and the particular flight vehicle-sensor combination of 109. The base-specific flight path data represents flight path segments or routes that form one or more trips where each trip originates from the particular base and travels to a sequence of facility clusters and scans the respective facility clusters and then returns back to the particular base. The flight path segments or routes for the one or more trips can be selected to cover the facility clusters for the particular base.

In embodiments, the optimization procedure of 117 can be formulated as a capacitated vehicle routing problem (VRP) for the collection of target-sites (the target clusters C_{L}) that are produced by block 115. That is, how many trips are required from the starting base B to serve each cluster in the set C_{L} and then return to the same base B within the total flying time of the vehicle. A suitable VRP solver (such as one following the Unified Tabu Search method described by Cordeau et al. in “A unified tabu search heuristic for vehicle routing problems with time windows”, Journal of the Operational Research Society (2001) 52, 928-936) can be used to address this problem. The VRP solver typically employs a definitive graph with vertices and associated vertex costs, edges between vertices and associated edge costs as well as capacity constraints. In embodiments, the facility clusters that are produced by block 115 can define the vertices of the graph, the time to travel between target sites (clusters) (which can be determined from the vehicle cruising speed) can define the edge costs in the graph, the scan times for scanning the one or more facilities in the clusters (which can be determined from the area of the cluster and the vehicle scan speed and other operational parameters of the vehicle and airborne sensor) can define the vertex costs in the graph, and vehicle range limits can define capacity constraints. Similarly, no-fly zone restrictions and other limitations stipulated by the constraint set C_{L} can be added as penalties on the non-compliant edges from the outset. The VRP problem can be stated in general terms as:

min W=VRP(Y|C_{L},V,S,B,C) Eqn. (5)

where, Y represents a set of routes (flight paths) each comprising a sequence of facility visits by index with design merit value W, C_{L} is the set of target clusters with determined scan time costs, V represents the vehicle, S is the sensor, B is the base and C is the set of constraints.

A cost matrix can be used to establish the edge costs (in terms of time) from the base or target site (cluster) to any other target site or base. The VRP solution Y, will yield the optimal number of trips along with their flight segments (routes) that minimize the overall time (and therefore distance) as a measure of the cost to complete the scanning task of the set of target clusters C_{L} with vehicle V fitted with sensor S from base B.

Note that as a vehicle is deemed to travel to target sites (clusters) at vehicle cruising speed but undertakes scan operations of the one or more facilities within a cluster at vehicle scan speed, cumulative time can be used as the measure of performance that also allows consideration of vehicle total hire time. However, distance, or some other metric, could also be used. Financial parameters (stemming from operator practices or due to prevailing cost models) can be applied to the (time-distance) solution produced by the optimization procedure above (block 125). The complete solution can be stored in a table or other data structure and can be accessed later for comparative purposes for different vehicle and sensor combinations (block 129).

In block 119, the base-specific flight path data and the overall scan time(s) for the sequence of clusters in the flights paths determined by the optimization procedure of block 117 provide a base-specific overall flight time, which represents the time cost to complete the scanning task of the facility clusters for the particular base.

In optional block 121, the operations of 113-119 can be repeated for one or more additional bases in the particular region such that base-specific flight path data (and the associated base-specific overall flight times provided in 119) cover all of the facilities in the particular region of 109.

In block 123, a total flight time for the particular flight vehicle-sensor combination of 109 can be determined by summing the base-specific overall flight times (as provided in 119) for the base(s) that cover the facilities in the particular region of 109.

In block 125, the total flight time of 123 for the particular flight vehicle-sensor combination and financial cost model parameters for the particular flight vehicle-sensor combination can be used to determine the total cost associated with scanning the facilities in the particular region of 109 using the particular flight vehicle-sensor combination of 109.

In block 127, the operations of 111-125 can be repeated for one or more additional flight vehicle-sensor combinations of the set of 107.

In block 129, one or more result parameters generated by the workflow (such as the total flight time and/or total cost) for different flight vehicle-sensor combinations can be evaluated to select one of the flight vehicle-sensor combinations of the set of 107. For example, it is contemplated that the flight vehicle-sensor combination selected in 129 has the lowest total flight time or lowest total cost as compared to the other flight vehicle-sensor combination in the set of 107.

In block 131, the base-specific flight path data as determined in 117 for the flight vehicle-sensor combination selected in 129 and those base(s) that cover the facilities in the particular region of 109 is collected.

In block 133, the flight vehicle and sensor selected in 129 can be flown along flight paths (routes) that correspond to the base-specific flight path data collected in 131, and the sensor is controlled during the flight to scan the facilities covered by the collected base-specific flight path data.

In block 135, the scan results for each facility are evaluated to detect methane emission. If methane emission is detected in block 135, the operations continue to block 137. Otherwise, the operations end for that facility.

In block 137, the facility is marked for further processing or component-level inspection/repair.

In block 139, the scan results for the facility are evaluated to determine if the detected methane emission is due to allowed emissions. For example, the time of the scanning of the facility can be evaluated to determine if it corresponds to time of known allowed emissions, such as liquid unloading. If the detected methane emission is due to allowed temporary, the operations continue to block 141; if not, the operations continue to block 143.

In block 141, the facility can be marked for a subsequent aerial scan and possibly perform component-level inspection and repair, if needed.

In block 143, the facility is marked for component-level inspection and repair.

In block 145, the component-level inspection of the facility is scheduled and performed, and repair of the component(s) or equipment of the facility can be performed if need be to mitigate fugitive methane emission from the facility. The component-level inspection can involve inspection of valves, flanges, tanks or other equipment or other components of the facility. In embodiments, the component-level inspection of block 145 can utilize portable technology that can effectively identify and locate methane emissions, such as a gas sniffer or an optical gas imager or other sensors. The repair of the equipment of the facility can use standard best practices. Optionally, the quality of the repair can be verified by inspecting the repaired equipment. In embodiments, the same portable technology used for the component-level inspection can be used to validate and verify the leak mitigation provided by the repaired equipment.

FIG. 2 illustrates an exemplary flight path planning solution produced as a result of the workflows described herein. The flight path planning solution is provided for scanning a set of facilities (e.g., well sites) in the Permian Basis of Texas using Lubbock airport as a base. The well sites are shown as dots distributed over the map of the Permian Basin around the Lubbock airport. The flight path segments or routes of the solution are shown as edges/lines. The flight path segments form four different trips (labeled trip 1, trip 2, trip 3, and trip 4) that originate and terminate at Lubbock airport (base). A set of three clusters that are part of trip 3 is shown in the expanded view window on the right-hand side of the page. The clusters are scanned by a scan pattern as shown in the expanded view window.

Note that various adaptations can be made to the workflows as described herein. For example, in carrying out the operations of the workflow of FIGS. 1A-1C, some or all of the optimization procedures described herein can be used determine the optimal flight plan (flight path data) for the facility scanning operations. In another example, if a particular airborne sensor is preferred, the workflow can be configured to optimize the selection of a flight vehicle (from number of possible flight vehicles) and the generation of the flight path that uses the selected flight vehicle and particular airborne sensor to scan the facilities of a desired region. In yet another example, if a particular flight vehicle is preferred, the workflow can be configured to optimize the selection of an airborne sensor (from number of possible airborne sensors) and the generation of the flight path that uses the particle flight vehicle and the selected airborne sensor to scan the facilities of a desired region. In still another example, if a particular flight vehicle-sensor combination is preferred, the workflow can be configured to optimize the generation of the flight path route that uses the particle flight vehicle—sensor combination to scan the facilities of a desired region. For example, such operations can involve the execution of blocks 111 to 121 of the workflow of FIGS. 1A-1C, while omitting operations (such as the iterative processing of blocks 123 to 129 over the possible flight vehicle—sensor combinations) that allow for selection of the optimal flight vehicle-sensor combination.

The description provided here focuses on emissions of natural gas from distributed upstream oil-gas facilities such as well sites, compressor stations, or other upstream facilities. However, the workflow can readily be adapted to plan aerial scanning for methane detection in or other oilfield equipment, including equipment in the midstream and downstream sectors, including at any point in delivery of gas up to the point of use.

Some of the methods and processes described above, can be performed by a processor. The term “processor” should not be construed to limit the embodiments disclosed herein to any particular device type or system. The processor may include a computer system. The computer system may also include a computer processor (e.g., a microprocessor, microcontroller, digital signal processor, or general-purpose computer) for executing any of the methods and processes described above.

The computer system may further include a memory such as a semiconductor memory device (e.g., a RAM, ROM, PROM, EEPROM, or Flash-Programmable RAM), a magnetic memory device (e.g., a diskette or fixed disk), an optical memory device (e.g., a CD-ROM), a PC card (e.g., PCMCIA card), or other memory device.

Some of the methods and processes described above, can be implemented as computer program logic for use with the computer processor. The computer program logic may be embodied in various forms, including a source code form or a computer executable form. Source code may include a series of computer program instructions in a variety of programming languages (e.g., an object code, an assembly language, or a high-level language such as C, C++, or JAVA). Such computer instructions can be stored in a non-transitory computer readable medium (e.g., memory) and executed by the computer processor. The computer instructions may be distributed in any form as a removable storage medium with accompanying printed or electronic documentation (e.g., shrink wrapped software), preloaded with a computer system (e.g., on system ROM or fixed disk), or distributed from a server or electronic bulletin board over a communication system (e.g., the Internet or World Wide Web).

Alternatively or additionally, the processor may include discrete electronic components coupled to a printed circuit board, integrated circuitry (e.g., Application Specific Integrated Circuits (ASIC)), and/or programmable logic devices (e.g., a Field Programmable Gate Arrays (FPGA)). Any of the methods and processes described above can be implemented using such logic devices.

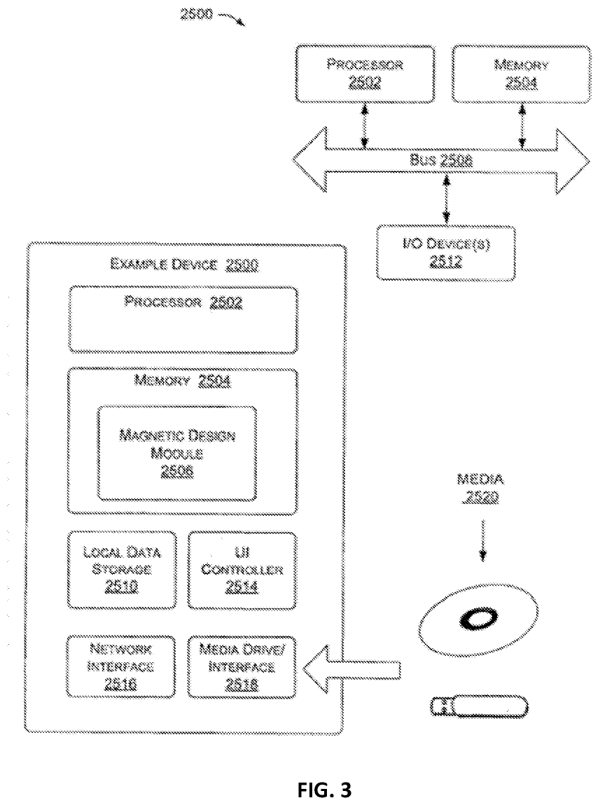

FIG. 3 illustrates an example device 2500, with a processor 2502 and memory 2504 that can be configured to implement various parts of the workflows and methods discussed in this disclosure. Memory 2504 can also host one or more databases and can include one or more forms of volatile data storage media such as random-access memory (RAM), and/or one or more forms of nonvolatile storage media (such as read-only memory (ROM), flash memory, and so forth).

Device 2500 is one example of a computing device or programmable device, and is not intended to suggest any limitation as to scope of use or functionality of device 2500 and/or its possible architectures. For example, device 2500 can comprise one or more computing devices, programmable logic controllers (PLCs), etc.

Further, device 2500 should not be interpreted as having any dependency relating to one or a combination of components illustrated in device 2500. For example, device 2500 may include one or more of a computer, such as a laptop computer, a desktop computer, a mainframe computer, etc., or any combination or accumulation thereof.

Device 2500 can also include a bus 2508 configured to allow various components and devices, such as processors 2502, memory 2504, and local data storage 2510, among other components, to communicate with each other.

Bus 2508 can include one or more of any of several types of bus structures, including a memory bus or memory controller, a peripheral bus, an accelerated graphics port, and a processor or local bus using any of a variety of bus architectures. Bus 2508 can also include wired and/or wireless buses.

Local data storage 2510 can include fixed media (e.g., RAM, ROM, a fixed hard drive, etc.) as well as removable media (e.g., a flash memory drive, a removable hard drive, optical disks, magnetic disks, and so forth).

One or more input/output (I/O) device(s) 2512 may also communicate via a user interface (UI) controller 2514, which may connect with I/O device(s) 2512 either directly or through bus 2508.

In one possible implementation, a network interface 2516 may communicate outside of device 2500 via a connected network.

A media drive/interface 2518 can accept removable tangible media 2520, such as flash drives, optical disks, removable hard drives, software products, etc. In one possible implementation, logic, computing instructions, and/or software programs comprising elements of module 2506 may reside on removable media 2520 readable by media drive/interface 2518.

In one possible embodiment, input/output device(s) 2512 can allow a user to enter commands and information to device 2500, and also allow information to be presented to the user and/or other components or devices. Examples of input device(s) 2512 include, for example, sensors, a keyboard, a cursor control device (e.g., a mouse), a microphone, a scanner, and any other input devices known in the art. Examples of output devices include a display device (e.g., a monitor or projector), speakers, a printer, a network card, and so on.

Various processes of present disclosure may be described herein in the general context of software or program modules, or the techniques and modules may be implemented in pure computing hardware. Software generally includes routines, programs, objects, components, data structures, and so forth that perform particular tasks or implement particular abstract data types. An implementation of these modules and techniques may be stored on or transmitted across some form of tangible computer-readable media. Computer-readable media can be any available data storage medium or media that is tangible and can be accessed by a computing device. Computer readable media may thus comprise computer storage media. “Computer storage media” designates tangible media, and includes volatile and non-volatile, removable and non-removable tangible media implemented for storage of information such as computer readable instructions, data structures, program modules, or other data. Computer memory includes, but are not limited to, RAM, ROM, EEPROM, flash memory or other memory technology, CD-ROM, digital versatile disks (DVD) or other optical storage, magnetic cassettes, magnetic tape, magnetic disk storage or other magnetic storage devices, computer storage media or any other tangible medium which can be used to store the desired information and data structures of the methods and workflows as described herein, and which can be accessed by a computer executing the operations of the methods and workflows as described herein.

The workflows and related data processing systems as described herein provide for flight path route planning for aerial detection (using airborne sensors mounted on flight vehicles). The aerial detection can be configured for remote detection of methane emission sources at distributed facilities. In other embodiments, the aerial detection can be configured for remote detection of emission sources other than methane, for aerial photography (using visual or other parts of the EM spectrum), etc. That is, the method is applicable in cases where surveys are possible with suitable airborne sensors, and does not limit the subsequent investigation step, if desired.

Although only a few examples have been described in detail above, those skilled in the art will readily appreciate that many modifications are possible in the examples without materially departing from this subject disclosure. Accordingly, all such modifications are intended to be included within the scope of this disclosure as defined in the following claims. In the claims, means-plus-function clauses are intended to cover the structures described herein as performing the recited function and not only structural equivalents, but also equivalent structures. Thus, although a nail and a screw may not be structural equivalents in that a nail employs a cylindrical surface to secure wooden parts together, whereas a screw employs a helical surface, in the environment of fastening wooden parts, a nail and a screw may be equivalent structures. It is the express intention of the applicant not to invoke 35 U.S.C. § 112, paragraph 6 for any limitations of any of the claims herein, except for those in which the claim expressly uses the words ‘means for’ together with an associated function.

What is claimed is:

1. A method of mitigating fugitive methane emission comprising:
scanning a plurality of facilities for fugitive methane emission using an airborne sensor; and
classifying the plurality of facilities based on results of the scanning.

2. The method of claim 1, further comprising:
selectively performing further inspection of at least one facility of the plurality of facilities for fugitive methane emission based on the classifying; and/or
selectively repairing at least one facility of the plurality of facilities based on the further inspection in order to mitigate fugitive methane emission.

3. The method of claim 1, further comprising:
building a map of the plurality of facilities.

4. The method of claim 1, further comprising:
determining a flight path for the scanning.

5. The method of claim 4, wherein:
the flight path is determined by minimizing flight time costs for the scanning.

6. The method of claim 4, wherein:
the flight path covers a set of facility clusters that are serviced by a respective base.

7. The method of claim 6, further comprising:
using a computer-implemented clustering method to identify the set of facility clusters that are serviced by the respective base; and
using a computer-implemented vehicle routing problem (VRP) solver to determine flight path data that represents the flight path that covers the set of facility clusters that are serviced by the respective base as output by the clustering method.

8. The method of claim 7, wherein:
the flight path data represents a trip that originates from the respective base and travels to a sequence of facility clusters that corresponds to the set of facility clusters and scans each facility in each facility cluster and returns back to the respective base.

9. The method of claim 1, wherein:
the airborne sensor comprises a laser-based sensor.

10. The method of claim 1, wherein:
the plurality of facilities are selected from the group consisting of well sites, compressor stations, and other upstream facilities.

11. The method of claim 1, wherein:
the airborne sensor is mounted to an aircraft selected from the group consisting of a drone, a helicopter, a fixed-winged airplane, or other aircraft or flight vehicle.

12. A method for planning aerial inspection of a plurality of facilities in a geographical region, the method comprising:
a) storing data that represents the plurality of facilities in the geographical region and data that represents at least one base in the geographical region, wherein the at least one base supports aerial inspection of the plurality of facilities in the geographical region;
b) selecting a particular base in the geographical region;
c) performing a clustering method on the data of a) to define cluster data representing a set of facility clusters in the geographical region that are associated with the particular base of b); and
d) processing the cluster data of c) to determine flight path data representing flight path segments that form a trip, wherein the trip originates at the particular base, travels to a sequence of facility clusters and scans each facility in each facility cluster, and returns back to the particular base, wherein the sequence of facility clusters of the trip corresponds to the set of facility clusters represented by the cluster data of c).

13. The method of claim 12, wherein:
the data of a) is stored in computer memory; and
the operations of c) and d) are performed by at least one processor.

14. The method of claim 12, wherein:
in d), the flight path data representing the flight segments of the trip is determined by minimizing flight time costs for the trip.

15. The method of claim 14, further comprising:
storing flight vehicle data that represents operational parameters for at least one flight vehicle, and storing sensor data that represents operational parameters for at least one airborne sensor;
wherein, in d) the flight time costs for the trip are based on the flight vehicle data and the sensor data.

16. The method of claim 12, further comprising:
repeating the operations of c) and d) for at least one additional base in the geographic region.

17. The method of claim 12, further comprising:
repeating the operations of c) and d) for different combinations of flight vehicle and airborne sensor that could be used for the aerial inspection.

18. The method of claim 17, wherein:
the different combinations of flight vehicle and airborne sensor have different flight vehicles.

19. The method of claim 17, wherein
the different combinations of flight vehicle and airborne sensor have different airborne sensors.

20. The method of claim 17, wherein
the different combinations of flight vehicle and airborne sensor have both different flight vehicles and different airborne sensors.

21. The method of claim 20, further comprising:
using the flight path data of d) to determine overall costs for the different combinations of flight vehicle and airborne sensor; and
evaluating the overall costs for the different combinations of flight vehicle and airborne sensor in order to select a particular combination of flight vehicle and airborne sensor that will be used for the aerial inspection.

22. The method of claim 21, wherein:
the overall costs for the different combinations of flight vehicle and airborne sensor are based on financial parameters for the different combinations of flight vehicle and airborne sensor.

23. The method of claim 21, further comprising:
using the particular combination of flight vehicle and airborne sensor and the flight path data of d) for the particular combination of flight vehicle and airborne sensor to perform the aerial inspection of the facilities in the geographical region.

24. The method of claim 12, wherein:
the clustering method of c) is a hierarchical multilevel clustering method.

25. The method of claim 12, wherein:
the clustering method of c) is applied to a filtered set of facilities that are associated with the particular base.

26. The method of claim 12, wherein:
the processing of d) uses a computer-implemented vehicle routing problem (VRP) solver to determine the flight path data.

27. The method of claim 26, wherein:
the VRP solver employs a graph with the facility clusters defined as vertices of the graph, time to travel between clusters at flight vehicle cruising speed defined as edge costs in the graph, scan times for scanning each facility in the clusters embedded as vertex costs in the graph, and vehicle range limits imposed as capacity constraints.

28. The method of claim 27, wherein:
no-fly zone restrictions and possibly other limitations are defined by a set of constraints that are added as penalties on non-compliant edges of the graph.

29. The method of claim 14, further comprising:
storing data representing a template scan pattern which is intended to be used in scanning one or more facilities in a respective cluster;
wherein the flight time costs include scanning costs for scanning the respective cluster which is based on the data representing the template scan pattern.

30. The method of claim 29, wherein:
the scanning costs for scanning the respective cluster is further based on parameters of a bounding box that covers the one or more facilities in the respective cluster.

31. The method of claim 14, wherein:
the flight time costs include scanning costs for scanning the one or more facilities in a respective cluster, which is based on optimization of the flight pattern for the one or more facilities of the respective cluster to minimize flight times for scanning the one or more facilities of the respective cluster.

32. The method of claim 14, further comprising:
storing data representing flight vehicle scan speed which is intended to be used in carrying out scanning one or more facilities in a respective cluster;
wherein the flight time costs include scanning costs for scanning one or more facilities in a respective cluster, which is based on flight vehicle scan speed in carrying out the scanning.

33. The method of claim 14, further comprising:
storing data representing flight vehicle cruise speed;
wherein the flight time costs are based on the flight vehicle cruise speed for the flight segments of the trip between the base to the sequence of facility clusters, between facility clusters, and back to the base.

34. The method of claim 14, wherein:
the flight time costs are based on at least one operational parameter of an airborne sensor.

35. The method of claim 34, wherein:
the at least one operational parameter is selected from the group consisting of scan swath, scan speed, scan radius, limit of detection, weight, cost, and deployment restrictions.

36. The method of claim 34, wherein:
the airborne sensor comprises a laser-based sensor.

37. The method of claim 14, wherein:
the flight time costs are based on at least one operational parameter of a flight vehicle.

38. The method of claim 37, wherein:
the at least one operational parameter is selected from the group consisting of cruise speed, fuel burn rate, fuel capacity, and turn rate.

39. The method of claim 37, wherein:
the flight vehicle is selected from the group consisting of a drone, a helicopter, and a fixed-winged airplane.

40. The method of claim 12, wherein:
the aerial inspection scans a plurality of facilities in the geographical region for fugitive emission of methane.

41. The method of claim 40, wherein:
the plurality of facilities are selected from the group including well sites, compressor stations, and other upstream facilities.

42. An apparatus comprising:
computer memory storing data that represents a plurality of facilities in the geographical region as well as at least one base in the geographic region, wherein the at least one base supports aerial inspection of the plurality of facilities in the geographical region; and
at least one processor configured to perform operations that involve
a) selecting a particular base in the geographical region;
b) performing a clustering method on the data stored in the computer memory to define cluster data representing a set of facility clusters in the geographical region that are associated with the particular base; and
c) processing the cluster data of b) to determine flight path data representing flight path segments that form a trip, wherein the trip originates at the particular base, travels to a sequence of facility clusters and scans each facility in each facility cluster, and returns back to the particular base, wherein the sequence of facility clusters of the trip corresponds to the set of facility clusters represented by the cluster data of b).

43. The apparatus of claim 42, wherein:
the processor determines the flight path data representing the flight path segments of the trip by minimizing flight time costs for the trip.

44. The apparatus of claim 42, wherein:
the aerial inspection scans a plurality of facilities in the geographical region for fugitive emission of methane.
